(19) United States
(12) Patent Application Publication
(10) Pub. No.: US 2025/0249083 A1
(43) Pub. Date: Aug. 7, 2025

(54) PHARMACEUTICAL PREPARATION FOR THE TREATMENT OF THE SYMPTOMS OF ADDICTION AND METHOD OF DIAGNOSING SAME

(71) Applicant: Curemark, LLC, Rye Brook, NY (US)

(72) Inventor: Joan M. Fallon, White Plains, NY (US)

(21) Appl. No.: 18/887,646

(22) Filed: Sep. 17, 2024

Related U.S. Application Data
(63) Continuation of application No. 17/551,942, filed on Dec. 15, 2021, now abandoned.
Application No. 17/551,942 is a continuation of application No. 16/281,937, filed on Feb. 21, 2019, now Pat. No. 11,235,038.
Application No. 16/281,937 is a continuation of application No. 15/593,121, filed on May 11, 2017, now Pat. No. 10,272,141.
Application No. 15/593,121 is a continuation of application No. 14/639,425, filed on Mar. 5, 2015, now Pat. No. 9,687,534.
Application No. 14/639,425 is a continuation of application No. 13/926,822, filed on Jun. 25, 2013, now Pat. No. 9,017,665.
Application No. 13/926,822 is a continuation of application No. 13/562,999, filed on Jul. 31, 2012, now Pat. No. 8,486,390.
Application No. 13/562,999 is a continuation of application No. 13/271,783, filed on Oct. 12, 2011, now Pat. No. 8,318,158.
Application No. 13/271,783 is a continuation of application No. 12/426,794, filed on Apr. 20, 2009, now Pat. No. 8,084,025.
(60) Provisional application No. 61/046,026, filed on Apr. 18, 2008.

(51) Int. Cl. A61K 38/54; A61K 38/46; A61K 38/47; A61K 38/48

(52) U.S. Cl. CPC A61K 38/54; A61K 38/465; A61K 38/47; A61K 38/4826; A61K 38/4873

(57) ABSTRACT

A therapeutic agent for the treatment of the symptoms of addiction and the method for preparing the therapeutic agent is disclosed. The therapeutic agent is a stable pharmaceutical preparation containing, but not limited to, digestive/pancreatic enzymes. The therapeutic agent may be manufactured by a variety of encapsulation technologies. Delivery of the therapeutic agent may be made orally, through injection, by adherence of a medicated patch or other method. Further, a method of using of a biomarker, the presence of chymotrypsin in the gastrointestinal tract to determine the presence of symptoms of addiction, and the likelihood of relapsing into addiction is disclosed.

### RELATED APPLICATIONS

This application is a continuation of U.S. application Ser. No. 17/551,942, filed Dec. 15, 2021, which is a continuation of U.S. application Ser. No. 16/281,937, filed Feb. 21, 2019, now U.S. Pat. No. 11,235,038, which is a continuation of U.S. application Ser. No. 15/593,121, filed May 11, 2017, now U.S. Pat. No. 10,272,141, which is a continuation of U.S. application Ser. No. 14/639,425, filed Mar. 5, 2015, now U.S. Pat. No. 9,687,534, which is a continuation of U.S. application Ser. No. 13/926,822, filed Jun. 25, 2013, now U.S. Pat. No. 9,017,665, which is a continuation of U.S. application Ser. No. 13/562,999, filed Jul. 31, 2012, now U.S. Pat. No. 8,486,390, which is a continuation of U.S. application Ser. No. 13/271,783, filed Oct. 12, 2011, now U.S. Pat. No. 8,318,158, which is a continuation of U.S. application Ser. No. 12/426,794, filed Apr. 20, 2009, now U.S. Pat. No. 8,084,025, which claims the benefit of U.S. Provisional Patent Application No. 61/046,026 filed on Apr. 18, 2008, each of which is incorporated by reference herein in its entirety.

### TECHNICAL FIELD

The invention relates to a treatment for the symptoms of addiction, and more particularly, to the use of digestive/pancreatic enzymes in the treatment of the symptoms of drug and alcohol addiction. The invention further relates to method of diagnosing the symptoms of addiction. More particularly, the invention further relates to measuring fecal chymotrypsin levels to diagnose the symptoms of drug and alcohol addiction and the possibility of relapse into drug and alcohol addiction.

### BACKGROUND OF THE DISCLOSURE

Addiction is a dependence on a behavior or substance that a person is powerless to stop. The term has been partially replaced by the word “dependence” for substance abuse. Addiction has been extended, however, to include mood-altering behaviors or activities. Some researchers speak of two types of addictions: substance addictions (for example, alcoholism, drug abuse, and smoking); and process addictions (for example, gambling, spending, shopping, eating, and sexual activity). There is a growing recognition that many addicts, such as polydrug abusers, are addicted to more than one sub-stance or process.

Addiction is one of the most costly public health problems in the United States. It is a progressive syndrome, which means that it increases in severity over time unless it is treated. Substance abuse is characterized by frequent relapse or return to the abused substance. Substance abusers often make repeated attempts to quit before they are successful.

In 1995 the economic cost of substance abuse in the United States exceeded $414 billion, with health care costs attributed to substance abuse estimated at more than $114 billion.

By eighth grade, 52% of adolescents have consumed alcohol, 41% have smoked tobacco, and 20% have smoked marijuana. Compared to females, males are almost four times as likely to be heavy drinkers, nearly one and a half more likely to smoke a pack or more of cigarettes daily, and twice as likely to smoke marijuana weekly. However, among adolescents these gender differences are decreasing. Although frequent use of tobacco, cocaine and heavy drinking appears to have remained stable in the 1990s, marijuana use increased.

In 1999, an estimated four million Americans over the age of 12 used prescription pain relievers, sedatives, and stimulants for “nonmedical” reasons during one month.

In the United States, 25% of the population regularly uses tobacco. Tobacco use reportedly kills 2 5 times as many people each year as alcohol and drug abuse combined. According to 1998 data from the World Health Organization, there were 1.1 billion smokers worldwide and 10,000 tobacco-related deaths per day. Furthermore, in the United States, 43% of children aged 2 to 11 years are exposed to environmental tobacco smoke, which has been implicated in sudden infant death syndrome, low birth weight, asthma, middle ear disease, pneumonia, cough, and upper respiratory infection.

Individuals going through alcohol rehabilitation or breaking drug addiction know the pain that chemicals can cause. All recovering alcoholics and drug users understand the toll that drug abuses take on the body, mind and emotions. Alcohol and drugs cause tremendous nutrient deficiencies as well as a need for very specific nutrients that are only found within nature's foods. These nutrients: support the liver, help the body detoxify, produce energy, rebuild vitamin and mineral levels, feed the brain and emotions, support the blood vessels, feed the nervous system, balance blood sugar.

One of the most obvious signs of drug and alcohol abuse is the depletion of the vitamin B complex. The challenge of addiction recovery can be made more difficult when coupled with nutritional deficiencies. The chronic depletion of vitamin B complex, for example, can lead to adrenal depletion as well. Since vitamin B complex provides cellular energy, nervous transmission, muscle health (contraction and relaxation), heart health, blood sugar metabolism, normal weight control, cellular regrowth and many functions related to emotional stability and thinking processes, this vitamin complex is absolutely necessary for replenishment. But vitamin B complex should only come from food, not from isolated or groups of vitamin pills. Similarly, the depletion of minerals such as calcium is very serious. Other important minerals include selenium, phosphorus, sulfur, zinc, potassium, and magnesium, which are all found in nature's raw vegetables.

Of course, the liver is one of the most injured organs in cases of alcoholism and drug abuse. The reason is because the liver is part of the digestive system and is used by the body to filter out and store toxins. When it is overtaxed, the liver can become fatty or damaged or both. It is critical that those going through drug or alcohol rehabilitation emphasize liver healing.

Alcohol blocks the absorption and breakdown of nutrients by damaging the cells lining the stomach and intestines, and by decreasing the amount of digestive enzymes secreted by the pancreas. For reasons that aren't yet known, the pancreas can become inflamed and leak digestive enzymes, which then attack the pancreas itself. Pancreatitis is extremely painful and can be fatal.

In view of such findings, there is need for a method of treating those exhibiting symptoms of drug and alcohol addiction.

No description in the Background section should be taken as an admission that such disclosure constitutes prior art to the instant invention.

### SUMMARY OF THE DISCLOSURE

The present invention is directed to the use of therapeutic agents in the treatment of the symptoms of drug and alcohol addiction and the method of preparing those agents. Further, the invention is directed to a method of diagnosing the symptoms of addiction and the possibility of relapse into drug and alcohol addiction.

More specifically, the present invention relates to stable pharmaceutical preparations containing, but not limited to, digestive/pancreatic enzymes, including, but not limited to, amylases, proteases, cellulase, papaya, papain, bromelain, lipases, chymotrypsin and hydrolases. This combination is made by, but not limited to: direct compression, microencapsulation, lipid encapsulation, wet granulation or other methods including the use of PROSOLV®, microencapsulation, lipid encapsulation technology, or other suitable technology. This technology can include the use of rapid dissolution (rapid dissolve), time release or other delivery methods including oral, injection, patch or other method. Further, the delivery of the enzymes can be in the form of a tablet, sprinkles, sachet, capsules, caplets or other compressed tablet delivery, or other oral delivery method.

Further, the invention is directed toward the use of a biomarker, the presence of chymotrypsin in the GI tract to determine a lack of protein digestion, nutrient absorption, and other related symptoms of drug and alcohol addiction.

It is a goal of the present invention to provide therapeutic agents for the treatment of the symptoms of drug and alcohol addiction and provide a method for preparing those agents.

Another goal of the present invention is to formulate stable pharmaceutical preparations containing, but not limited to, digestive/pancreatic enzymes including, but not limited to, amylases, proteases, cellulase, papaya, papain, bromelain, lipases, chymotrypsin, and hydrolases. Yet another goal of the present invention is to make a combination of

digestive/pancreatic enzymes utilizing, by but not limited to: direct compression, microencapsulation, lipid encapsulation, wet granulation or other methods including the use of PROSOLV®, and other known excipients and additives to accomplish microencapsulation, lipid encapsulation, direct compression, wet or dry granulation or other suitable technology.

A further goal of the present invention is to deliver the preparation by means, which can include the use of rapid dissolution (rapid dissolve), time release, or other delivery methods including oral, injection, patch, or other method. Further, the delivery of the enzymes may be in the form of a tablet, capsule, sprinkles, sachet, or other oral delivery method.

An additional goal of the invention is to demonstrate the use of fecal chymotrypsin as a prognosticative indicator of the presence of the symptoms of drug and alcohol addiction, or the likelihood of an individual to relapse into drug and alcohol addiction.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### DETAILED DESCRIPTION

The individual who is diagnosed as alcoholic or as substance abuse addicted is administered a fecal chymotrypsin test where the level of the enzyme chymotrypsin is measured. The individual is then given an effective amount of pancreatic/digestive enzymes if the fecal chymotrypsin level is below 8.4 U/mg. This level is considered abnormal when compared to an individual without alcoholism or substance abuse addiction, or who is not at risk for such an addiction, or having been addicted at any time in the past, or who has other protein digestion problems.

The fecal chymotrypsin levels may be measured in someone at risk for becoming an alcoholic or substance abuse addicted or who has had a history of alcoholism or substance abuse and who may again become an alcoholic or substance abuse addicted. The steps involve the following: taking a stool sample from the individual to be diagnosed, measuring the level of fecal chymotrypsin in the stool sample, and comparing that level to an individual who does not have alcoholism, a substance abuse problem or other protein digestion problem. When the level is low, less than 8.4 U/mg, an effective amount of pancreatic enzymes is administered to the individual.

The invention may be used as the or one of the components of an alcohol treatment or substance abuse treatment program for an active alcoholic or substance abuse addict. The invention may also be utilized to keep someone at risk for becoming an alcoholic or substance abuser or it may be used to prevent someone from relapse into addiction. The pancreatic/digestive enzymes may be given to prevent addiction, such as alcoholism or substance abuse, to help someone who is presently an addict such as an alcoholic or drug addict, or to someone who is at risk of relapse. The enzymes may be administered as a result of a lowered fecal chymotrypsin level.

Pancreatic/digestive enzymes may be administered to those who are presently battling active addiction such as alcoholism or drug abuse. They may be given to those who are not actively addicted, but who have been addicted at another time and who are at risk for becoming an addict, such as an alcoholic, drug addict or other substance abuse addict. They may also be utilized for those who are deemed at risk for addiction, such as alcoholism, due to family history or other historical events, such as severe stress or other factors placing the individual at risk.

In one embodiment, a stable preparation of digestive/pancreatic enzymes is formed into a dosage formulation containing a therapeutically effective amount of a protease, an amylase, and/or a lipase. The formulation may include additional enzymes, such as pancreatin, chymotrypsin, trypsin, papain and/or papaya. Other combinations of digestive enzymes may also be used. These enzymes can be in the form of animal or plant derivatives, natural or synthetic.

The following outlines a formulary for digestive/pancreatic enzymes for treating the symptoms of addiction: Amylase 10,000-60,000 U.S.P., Protease 10,000-70,000 U.S.P., Lipase 4,000-30,000 U.S.P., Pancreatin 2,000-6,000 U.S.P., Chymotrypsin 2-5 mg, Trypsin 60-100 mg, Papain 3,000-10,000 U.S.P. units/mg, Papaya 30-60 mg.

The dosage formulation may be administered by an oral preparation including, but not limited to, an encapsulated tablet, mini-tabs, microcapsule, mini-capsule, time released capsule, sprinkle or other methodology. In one embodiment, the oral preparation is encapsulated using PROSOLV® technology. Alternatively, the oral preparation may be encapsulated using enteric coating, lipid encapsulation, direct compression, dry granulation, wet granulation, and/or a combination of these methods.

Fecal chymotrypsin is a sensitive, specific measure of proteolytic activity. Normal levels of chymotrypsin are considered be greater than 8.4 U/gram. Decreased values (less than 8.4 U/gram) suggest diminished pancreatic output (pancreatic insufficiency), hypoacidity of the stomach or cystic fibrosis. Elevated chymotrypsin values suggest rapid transit time, or less likely, a large output of chymotrypsin from the pancreas.

For the fecal chymotrypsin test, a stool sample is collected from each of the subjects. Each stool sample is analyzed using an enzymatic photo spectrometry analysis to determine the level of fecal chymotrypsin in the stool. Alternatively, other methods, such as the colorimetric method, use of substrates, use of assays, and/or any other suitable method may be used to measure the fecal chymotrypsin levels. The levels of fecal chymotrypsin in the samples of the individuals to be diagnosed are compared to the levels of fecal chymotrypsin in an individual who does not have alcoholism, a substance abuse problem or other protein digestion problem to determine if the individual being diagnosed would benefit from the administration of digestive enzymes.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A method for treating an addiction in a subject in need thereof, the method comprising administering to the subject an effective amount of a pharmaceutical preparation digestive enzymes to the subject, whereby the subject is treated.

2. The method of claim 1, wherein the digestive enzymes comprise an amylase, a lipase, a protease, or a combination thereof.

3. The method of claim 2, wherein the digestive enzymes comprise the amylase, the lipase, and the protease.

4. The method of claim 1, wherein the digestive enzymes further comprise a chymotrypsin, a trypsin, a papaya, a papain, or a combination thereof.

5. The method of claim 1, wherein the digestive enzymes are animal enzymes, plant enzymes, synthetic enzymes, or a combination thereof.

6. The method of claim 1, wherein the digestive enzymes are provided as pancreatin.

7. (canceled)

8. The method of claim 1, wherein the pharmaceutical preparation is a dosage formulation selected from the group consisting of a pill, a tablet, a capsule, a mini-tab, a sprinkle, and a combination thereof.

9. The method of claim 2, wherein the amylase is present in the pharmaceutical preparation in an amount of from about 10,000 to about 60,000 U.S.P. units/mg.

10. The method of claim 2, wherein the protease is present in the pharmaceutical preparation in an amount of from about 10,000 to about 70,000 U.S.P. units/mg.

11. The method of claim 2, wherein the lipase is present in the pharmaceutical preparation in an amount of from about 4,000 to about 30,000 U.S.P. units/mg.

12. The method of claim 4, wherein the chymotrypsin is present in the pharmaceutical preparation in an amount of from about 2 to about 5 mg.

13. The method of claim 4, wherein the papain is present in the pharmaceutical preparation in an amount of from about 3,000 to about 10,000 U.S.P. units/mg.

14. The method of claim 4, wherein the papaya is present in the pharmaceutical preparation in an amount of from about 30 to about 60 mg.

15. The method of claim 4, wherein the trypsin ranges is present in the pharmaceutical preparation in an amount of from about 60 to about100 mg.

16. The method of claim 6, wherein the pancreatin is present in the pharmaceutical preparation in an amount of from about 2,000 to about 6,000 U.S.P. units/mg.

17. The method of claim 1, wherein a symptom of the addiction is ameliorated.

18. The method of claim 17, wherein the symptom of the addiction is a lack of protein digestion, a lack of nutrient adsorption, or a combination thereof.

19. The method of claim 1, wherein the digestive enzymes are encapsulated with an enteric coating or a lipid coating.

20-31. (canceled)

32. A pharmaceutical preparation for treating a subject having an addiction, the pharmaceutical preparation comprising an effective amount of digestive enzymes.

33. A method of diagnosing an addiction in a subject, comprising measuring a level of fecal chymotrypsin in a stool sample obtained from the subject using an enzymatic photo spectrometry analysis, comparing the level of fecal chymotrypsin in the stool sample to a level of fecal chymotrypsin in a stool sample obtained from healthy subject, and diagnosing the subject as having an addiction when the subject has a level of fecal chymotrypsin of less than 8.4 U/mg.

34. (canceled)
